(19) United States
(12) Patent Application Publication
(10) Pub. No.: US 2013/0039980 A1
(43) Pub. Date: Feb. 14, 2013

(54) Time Release Capsule for Beverage

(71) Applicant: Alejandro I. Olmos, Chula Vista, CA (US)

(72) Inventor: Alejandro I. Olmos, Chula Vista, CA (US)

(21) Appl. No.: 13/570,066

(22) Filed: Aug. 8, 2012

Related U.S. Application Data
(60) Provisional application No. 61/521,978, filed on Aug. 10, 2011.

(51) Int. Cl. A23L 2/52; A61K 9/52

(52) U.S. Cl. 424/457; 426/89

(57) ABSTRACT

This invention relates to time-release capsules administered in beverage preparations. The present invention may be used for administration of delayed-release flavor or color enhancement of the beverage preparation. It may also be used for the oral administration of pharmaceutical compounds, vitamins, or other active compounds, including herbal ingredients, to a person or animal in a liquid beverage. This invention solves several problems associated with the blending of complex drinks and taste habituation wherein the taste intensity of a drink decreases with time. It also solves several practical problems associated with administration of compounds to a person or veterinary patient, including reduction of efficacy due to manufacturing issues, suboptimal dissolution in the digestive system, and administration of an unpleasant oral compound in a form that is displeasing to taste.

This invention claims the benefit of U.S. Provisional Application No. 61/521,978 with the title, “Time Release Capsule for Beverage,” which was filed on Aug. 10, 2011, and is hereby incorporated by reference. Applicant claims priority to that provisional application pursuant to 35 U.S.C. §119(e)(i).

### FIELD OF THE INVENTION

This invention relates to time-release capsules administered in beverage preparations. The present invention may be used for administration of delayed-release flavor or color enhancement of the beverage preparation. It may also be used for the oral administration of pharmaceutical compounds, vitamins, or other active compounds, including herbal ingredients, to a person or animal in a liquid beverage.

### INCORPORATION BY REFERENCE

The following patent applications have been incorporated by reference: US patent 2010/0136183 by Gonus et al., US patent application 2009/0291121 by HAAS et al., US patent application 207/0071808 by Janik et al., US application 2010/0212507 by Hester et al., and US application 2005/0118268 by Percel et al.

The following US patents have been incorporated by reference. U.S. Pat. No. 6,902,751 by Schleifenbaum et al., U.S. Pat. No. 5,958,502 y Fulger et al., U.S. Pat. No. 5,709,895 by Tanaka et al. U.S. Pat. No. 5,601,865 by Fulger et al., U.S. Pat. No. 5,399,368 by Garwood et al., U.S. Pat. No. 4,232,047 by Sair et al., U.S. Pat. No. 3,431,339 by Gyarmathy et al., U.S. Pat. No. 2,004,957 by Messner, U.S. Pat. No. 5,021,249 by Bunick et al., U.S. Pat. No. 4,689,235 by Barnes et al., U.S. Pat. No. 6,204,243 by Posanski, U.S. Pat. No. 5,401,512 by Rhodes et al., U.S. Pat. No. 6,663,888 by Percel et al., U.S. Pat. No. 6,500,454 by Percel et al., and U.S. Pat. No. 7,048,945 by Percel et al.

### BACKGROUND

The preparation of a beverage with ingredients that retain their flavor over a long period of time or retain a relatively mixed or homogeneous composition through the drinking process can be complicated. Additionally, while it may be desirable to orally administer pharmaceutical compounds, vitamins, or other active compounds, including herbal ingredients, in a liquid beverage, the variety of ingredients found in these beverages and the potential effect they may have on the compound being administered has prevented their use for this purpose. This is further complicated by the fact that many beverages contain a variety of ingredients with different densities that may be hydrophobic or hydrophilic substances, further adding to the difficulty of maintaining a near homogeneous mix of beverage ingredients over a period of time.

For instance, the blending of a mixed drink such as coffee or tea requires the preparer to mix together a variety of different ingredients. There is a large a number of coffee recipes, each requiring different ingredients, including but not limited to caffe Americano, cafait au lait, café bombon, cafémélange, café mocha, Ca phe sua da, café cortado, eiskaffee, espresso Romano, flat white, Indian filter coffee, Kopi sussu, nachiato, mazagran, mochasippi, Tukish coffee, Vienna coffee, and yuanyang.

Furthermore there are a number of coffees based on liquors including but not limited to Irish coffee (Whiskey), Brandy coffee (Brandy), keoke coffee (Brandy and Kahlúa), English coffee (Gin), calypso coffee (Tia Maria or Kahlúa and Rum), Jamaican coffee (Tia Maria & Rum), shin shin coffee (Rum), Baileys Irish cream coffee, monk's coffee (Bénédictine), Seville coffee (Cointreau), witch's coffee (Strega), Russian coffee (Vodka), priest coffee (Brennivín), Corfu coffee (Koum Quat liquor), kaffee fertig (coffee with Swiss prune schnapps), caffè corretto (that is an Italian beverage, consisting of a shot of espresso “corrected” with a shot of liquor, usually grappa, brandy or sambuca.). Coffee is sold and used in mixed drinks in the form of beans, ground beans, or in powders (instant).

There are a large number of tea recipes including but not limited to white tea, yellow tea, green tea, oolong tea, black tea, pu'er tea, and a much larger number of herbal teas. Tea and coffee additives can include sweeteners such as honey, and flavors such as mint. Tea comes in leaves, in bags or in powder.

Many other beverages exist beside mixed drinks that include coffees and teas, which require their own ingredients and are sold in their own unique packages. These include but are not limited to beer, wine, sangria, cider, hot cider, hot chocolate, holicks, etc.

Sometimes the preparation of a multi-ingredient beverage requires that a base liquid be added such as, but not limited to water, milk, soda water, wine, cider, beer, ethyl alcohol, and fruit juice.

The large range of ingredients used in beverage preparation presents a problem should the beverage also contain ingredients designed for long-term flavor delivery, color maintenance, vitamin augmentation, or delivery of pharmaceutical or herbal compounds.

In addition to the complexity of beverage preparation is the problem of loss of taste through the phenomenon of habituation: when a sense is exposed to a stimulus for a period of time, it loses its sensitivity to that stimulus. More particularly, a drink's flavor that may appear to be intense initially loses some of its intensity after the taste buds become used to that flavor. The prior art does not adequately address this problem.

Additionally, many vitamins, pharmaceutical compounds, or other active compounds including herbal ingredients might be added to a beverage for the purpose of providing ingredients that should be processed in the stomach during digestion or otherwise broken down and absorbed through the intestine in order to become efficacious. However, the prior art does not provide a method for effective delivery of these ingredients, using an oral delivery method involving a liquid beverage, in a manner that protects the relevant ingredient so that it is processed and absorbed to maximize exposure to and effectiveness of a delivered compound.

It is therefore one object of this invention to simplify the task of complex drink preparation. It is another object of the invention to mitigate the phenomenon of habituation and sustain a drink's taste intensity. Additionally, the invention provides for delayed-release drink color enhancement. The invention may also be used for the oral administration of pharmaceutical compounds, vitamins, or other active compounds including herbal ingredients to a person or veterinary patient in a liquid beverage.

Further features, aspects, and advantages of the present invention over the prior art will be more fully understood when considered in light of the following detailed description and claims.

### BRIEF DESCRIPTION OF THE DRAWINGS

FIG. 1 illustrates a capsule containing inner bodies.

FIG. 2 illustrates flavoring habituation curves with a peak immediately after the introduction of a new flavor, followed by a slow decay.

FIG. 3 shows the capsule matrix and the inner bodies layered in a stack.

### SUMMARY OF THE INVENTION

The phenomenon of habituation is well known and well documented. It refers to a type of non-associative learning in which repeated exposure to a stimulus leads to decreased response to it. This phenomenon is responsible for after-images seen in the eyes after the retina has been exposed to an image. The same phenomenon applies to other senses, in particular, the sense of taste: The second sip of a drink is usually less “tasty” or stimulating to the taste buds than the first. This invention mitigates the habituation phenomenon and sustains the intensity of a drink tasting experience by varying or modulating the taste of a beverage, over time. It utilizes time-release technologies involving capsules to expose the taste buds to flavors in a drink over an extended time period, thereby exciting the sense of taste over a longer period of time.

In addition, this invention provides an added practical benefit in that it streamlines the task of the beverage preparer by combining, into a single capsule, a number of beverage ingredients that may be deposited into the liquid base for faster and easier preparation of mixed drinks

In one aspect, the invention comprises a capsule filled with a matrix that contains (sequestered inside) the primary flavor, which suffuses through the beverage after the matrix has dispersed or dissolved in the beverage. Included inside this matrix are additional flavors captured in inner bodies that are designed to dissolve at later time intervals after they are released from the matrix. These inner bodies contain secondary, tertiary, and additional flavors that release after the primary beverage flavor response of the taste buds has subsided. In another beneficial aspect of the invention, a large number of inner bodies sequestered in a capsule produce a more complex flavor-time profile for the person drinking the beverage—essentially a music of flavors.

In this aspect, the invention comprises a capsule for delayed release of one or more flavor ingredients in a beverage, the time releases occurring in multiple stages due to the gradual degradation or dissolution of the capsule constituents, which results in a delayed release of flavor trapped within the capsule. The capsule comprises:
1) a shell composed of material soluble in the aqueous beverage when a predetermined condition is met. Examples of such condition include a temperature level or shaking of the liquid. The dissolution of the shell starts the multi-stage capsule degradation process.
2) a liquid, solid, or powder matrix material soluble in the aqueous beverage. The dissolution of the matrix occurs in the second stage, after the shell has dissolved sufficiently to expose the matrix to the liquid.
3) inner bodies inserted, embedded, or floating in the matrix. The inner bodies are configured to dissolve in the aqueous beverage in stages subsequent to the first and second stages, and after they are released from the matrix.

The shell, matrix, and inner bodies may optionally contain additional ingredients that are released into the beverage in corresponding stages. The invention includes embodiments wherein a variety of different types of inner bodies are assembled in coating layers, each layer having a predetermined rate of dissolution that is designed to achieve the desired time-release profile for the capsule's constituent ingredients. These additional inner bodies may include color enhancers that, when released into the beverage, change the hue of the beverage over time.

The invention also includes capsule embodiments designed for oral administration of pharmaceutical compounds, vitamins, or other active compounds including herbal ingredients to a person or veterinary patient in a liquid beverage, according to a predetermined time profile. These compounds may be placed in inner bodies that are inserted, embedded, or floating in the matrix. Because of the ingredients of these inner bodies are exposed to liquid and processed at a later time period, this time release profile is advantageous for the delivery of compounds that should be processed in the stomach or digestive track to be efficacious.

### DETAILED DESCRIPTION OF THE INVENTION

The present invention involves a capsule that contains the ingredients required to prepare a blended beverage that includes at least one time-release component. When the capsule is dissolved in liquid such as but not limited to water, milk, alcohol or juice, flavors, colors, compounds and other constituents of the capsule are released at various time intervals.

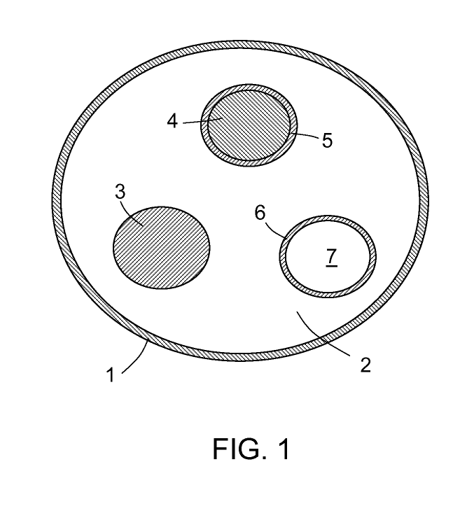

The invention shown in FIG. 1 comprises the following components:
1) A water-soluble capsule shell 1.
2) A matrix 2 material held by the capsule shell 1, which can be a solid, liquid, paste, gel, or powder.
3) At least one inner body 3, 5, and 6 that is inserted, embedded, or floating in the matrix.
4) At least one additional ingredient contained within inner bodies, which can include flavors, color, and pharmaceutical compounds, vitamins, or other active compounds including herbal ingredients.

The water-soluble capsule shell 1 of FIG. 1 is made of a material that dissolves easily in water and is well known in the art as a time-release capsule shell, such as hydroxymethyl cellulose. The thickness of shell 1 depends on its desired usefulness in a beverage delivery system and on how quickly the capsule is designed to dissolve in liquid preparations. The outside diameter of a capsule of the invention typically measures between 1½-3½ inches and the walls of the capsule shell 1 typically range from 1/16 to ⅛ of an inch thick. The composition of shell 1 is selected to be soluble in liquid when a predetermined condition is met, such as certain time duration, a predetermined temperature, or a certain amount of agitation of the beverage it has been added to. An inner body 3, 5, and 6 typically ranges from ⅛ to ¾ of an inch in its outer diameter. These inner bodies are designed to fit well within a capsule and its constituent matrix. Optionally, these inner bodies are coated with a material that dissolves when exposed to liquid, such as hydroxymethyl cellulose.

For example, if a hot beverage is prepared, the capsule wall may optionally begin to dissolve around 88° F. Alternatively, the walls may be designed to break open when the liquid it's contained within is violently shaken. In yet another variation of the present invention, the capsule wall is thick and only starts to significantly dissolve after being exposed to liquid for at least 5 minutes.

The matrix 2 of FIG. 1 is composed of material that can easily dissolve or become a homogeneous mixture in liquid. It may provide the beverage with its initial flavor and color and may typically comprise 25% to 75% sweetener and correspondingly, 75% to 25% flavor. For example sugar and milk, sugar and mango, sugar and vanilla may be used in the capsule matrix as a sweetener that is released into the beverage upon dissolution.

The matrix 2 of FIG. 1 holds at least one inner body 3, 5, and 6, which is designed to hold additional flavors and colors. These inner bodies dissolve in the liquid as the matrix releases them; this is the second stage of capsule dissolution in this timed-release process. The inner bodies' dissolution rate can be controlled or driven by the size and shape of the inner bodies, by the content and amount of matrix that holds them, and by the thickness and type of coatings on the capsule or on the inner bodies directly. FIG. 1 shows an inner body 3 with uniform density. It also shows another inner body 4, which is covered by a coat 5 designed to slow down its dissolution. The figure also illustrates a vesicule type inner body 6 that contains a liquid 7. A large number and variety of inner bodies may be incorporated within the capsule of the present invention.

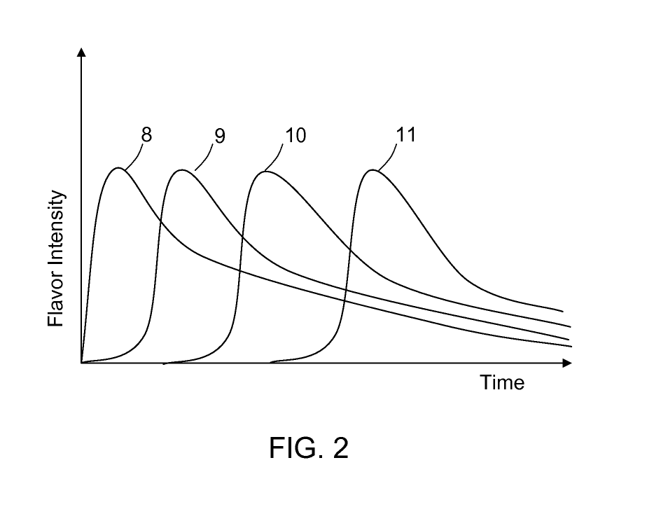

FIG. 2 illustrates how the phenomenon of habituation reduces the intensity of the flavor response over time. As a first flavor 8 is presented to the palate, its intensity peaks after the initial contact, and then decreases over time as the contact continues. A second flavor 9, followed by a third 10, and a fourth 11, are presented to the palate—all flavors following the same pattern of peaking and decaying according to the palate reaction. This invention intensifies the taste sense and flavor experienced from the drinking experience because the beverage is releasing flavors (whether consistent or different) over time. A larger number of inner bodies containing flavors means that the beverage containing one or more capsules of the present invention yields a more complex time-flavor profile—essentially a music of flavors. Flavors carried by inner bodies include but are not limited to, mint, vanilla, anis, coffee, chocolate, salt, pepper, lemon oil, orange oil, walnut oil, nut oils, etc.

The capsule of FIG. 1 may also be used to deliver and release color in the beverage over time. Thus, a beverage may be prepared that changes color over time, as the color is released from the capsule. The color may optionally reside in the matrix 2 of FIG. 1 or the inner bodies. Color may also be mixed with flavor, liquid, or other compounds within individual inner body 5 and 6.

Additional, the capsule of FIG. 1 may also be used to deliver and release pharmaceutical compounds, vitamins, or other active compounds, including herbal ingredients, to a person or animal through oral administration in a beverage, over time. Thus, a beverage may be prepared that releases compounds in the inner bodies during digestion in the stomach or in the intestine, after the capsule and matrix degrade and release the inner bodies. The present invention may be used for the oral administration of compounds to a person or animal in a liquid beverage.

### EXAMPLE 1

A capsule designed to deliver a flavored coffee beverage by dissolving it in hot water.

Capsule wall

| | |
|---|---|
| Magnesium stearate | 37% |
| Hydoxymethyl cellulose | 12% |
| Glycerin | 11% |
| Gelatin | 22% |
| Stearic acid | 6% |
| Coloring | 4% |
| Water | 8% |
| Total | 100% |

### Matrix

| | |
|---|---|
| Sugar | 40% |
| Milk | 36% |
| Vegetable oil | 10% |
| Vitamin A | 8% |
| Coloring (white/brown) | 4% |
| Water | 2% |
| Total | 100% |

### #1 Inner Body Coating

| | |
|---|---|
| Magnesium stearate | 52% |
| Hydoxymethyl cellulose | 12% |
| Glycerin | 8% |
| Gelatin | 14% |
| Coloring | 6% |
| Water | 8% |
| Total | 100% |

### #1 Inner Body Content

| | |
|---|---|
| Natural Hazelnut | 80% |
| Vitamin B12 | 8% |
| Coloring (Dark/Brown) | 2% |
| Water | 4% |
| Preservatives | 6% |
| Total | 100% |

### #2 Inner Body Coating

| | |
|---|---|
| Magnesium stearate | 64% |
| Hydoxymethyl cellulose | 12% |
| Glycerin | 10% |
| Coloring | 6% |
| Water | 8% |
| Total | 100% |

### #2 Inner Body Content

| | |
|---|---|
| Artificial Hazelnut | 80% |
| Nutrients & Vitamin B6 | 8% |
| Coloring (Dark/Brown) | 2% |
| Water | 4% |
| Preservatives | 6% |
| Total | 100% |

### #3 Inner Body Coating

| | |
|---|---|
| Magnesium stearate | 44% |
| Hydoxymethyl cellulose | 20% |
| Glycerin | 6% |
| Gelatin | 16% |
| Coloring | 6% |
| Water | 8% |
| Total | 100% |

### #3 Inner Body Content

| | |
|---|---|
| Coffee | 80% |
| Vitamin B2 | 8% |
| Coloring (Dark/Brown) | 2% |
| Water | 4% |
| Preservatives | 6% |
| Total | 100% |

### EXAMPLE 2

A capsule designed to deliver a mango-flavored water, with high vitamin content, in an energy drink by dissolving the capsule in cold water.

### Capsule Wall

| | |
|---|---|
| Magnesium stearate | 37% |
| Hydoxymethyl cellulose | 12% |
| Glycerin | 11% |
| Gelatin | 22% |
| Stearic acid | 6% |
| Coloring | 4% |
| Water | 8% |
| Total | 100% |

### Capsule Matrix

| | |
|---|---|
| Dextrose, Sugar | 40% |
| Guar Gum | 8% |
| Energy Blend, Taurine, Glucuronolactone, Malic acid, N-acetyl | 46% |
| L-trysine, Guarana, Caffeine | |
| 4% Yellow coloring | 4% |
| Water | 2% |
| Total | 100% |

### #1 Inner Body Coating

| | |
|---|---|
| Magnesium stearate | 52% |
| Hydoxymethyl cellulose | 12% |
| Glycerin | 6% |
| Gelatin | 14% |
| Nutrients | 6% |
| Coloring | 6% |
| Water | 4% |
| Total | 100% |

### #1 Inner Body Content

| | |
|---|---|
| Natural Mango | 80% |
| Vitamin B12 | 8% |
| Coloring (orange/yellow) | 2% |
| Water | 4% |
| Preservatives | 6% |
| Total | 100% |

### #2 Inner Body Coating

| | |
|---|---|
| Magnesium stearate | 64% |
| Hydoxymethyl cellulose | 12% |
| Glycerin | 10% |
| Coloring | 6% |
| Water | 8% |
| Total | 100% |

### #2 Inner Body Content

| | |
|---|---|
| Artificial Mango | 80% |
| Vitamin B6 | 8% |
| Coloring (orange/yellow) | 2% |
| Water | 4% |
| Preservatives | 6% |
| Total | 100% |

### #3 Inner Body Coating

| | |
|---|---|
| Magnesium stearate | 44% |
| Hydoxymethyl cellulose | 20% |
| Glycerin | 6% |
| Gelatin | 14% |
| Coloring | 8% |
| Water | 8% |
| Total | 100% |

### #3 Inner Body Content

| | |
|---|---|
| Artificial Pine Apple | 80% |
| Vitamin C | 8% |
| Coloring orange/yellow | 2% |
| Water | 4% |
| Preservatives | 6% |
| Total | 100% |

### EXAMPLE 3

A capsule designed to deliver an alcoholic beverage containing vitamins and flavors, by dissolving it in an alcoholic liquid, such as vodka.

### Capsule Walls

| | |
|---|---|
| 37% Magnesium stearate | 37% |
| 12% Hydoxymethyl cellulose | 12% |
| 11% Glycerin | 11% |
| 22% Gelatin | 22% |
| 6% Stearic acid | 6% |
| 6% Coloring | 6% |
| 6% Water | 6% |
| Total | 100% |

### Capsule Matrix

| | |
|---|---|
| Lemon Juice | 10% |
| Lemon oil | 36% |
| Sodium Bensoate | 10% |
| Vitamin B-Complex | 19% |
| Vitamin B-12 | 19% |
| Coloring (yellow) | 4% |
| Water | 2% |
| Total | 100% |

### #1 Inner Body Coating

| | |
|---|---|
| Magnesium stearate | 52% |
| Hydoxymethyl cellulose | 12% |
| Glycerin | 6% |
| Gelatin | 14% |
| Coloring | 8% |
| Water | 8% |
| Total | 100% |

### #1 Inner Body Content

| | |
|---|---|
| Salt | 80% |
| Coloring (white/red) | 4% |
| Water | 8% |
| Preservatives | 8% |
| Total | 100% |

### #2 Inner Body Coating

| | |
|---|---|
| Magnesium stearate | 64% |
| Hydoxymethyl cellulose | 12% |
| Glycerin | 8% |
| Coloring | 8% |
| Water | 8% |
| Total | 100% |

### #2 Inner Body Content

| | |
|---|---|
| 80% Peppers | 80% |
| 6% Coloring (white/red) | 6% |
| 8% Water | 8% |
| 6% Preservatives | 6% |
| Total | 100% |

### #3 Inner Body Coating

| | |
|---|---|
| 44% Magnesium stearate | 44% |
| 20% Hydoxymethyl cellulose | 20% |
| 6% Glycerin | 6% |
| 16% Gelatin | 16% |
| 6% Coloring | 6% |
| 8% Water | 8% |
| Total | 100% |

### #3 Inner Body Content

| | |
|---|---|
| 80% Vinegars | 80% |
| 6% Coloring (white/red) | 6% |
| 8% Water | 8% |
| 6% Preservatives | 6% |
| Total | 100% |

Even though the above narrative describes the time release of flavors found in inner bodies in a drink, the same approach may also be used to deliver a timed release of nutrients, such as vitamins or herbs, compounds, or visual effects, such as colors. In one aspect of the invention, a change in color may be synchronized with the change in flavor to indicate, for example, the point of maximum flavor intensity for a particular flavor. These changes are driven by release of the inner bodies from the capsule matrix and degradation of any additional protective coating located directly around the inner bodies, which allows their ingredients to mix with the beverage.

In another embodiment of the present invention, the capsule contents, including at least some of the inner bodies, provide the person or veterinary patient with an oral dosage or administration of pharmaceutical compounds, vitamins, other active compounds, or herbal ingredients in a liquid beverage.

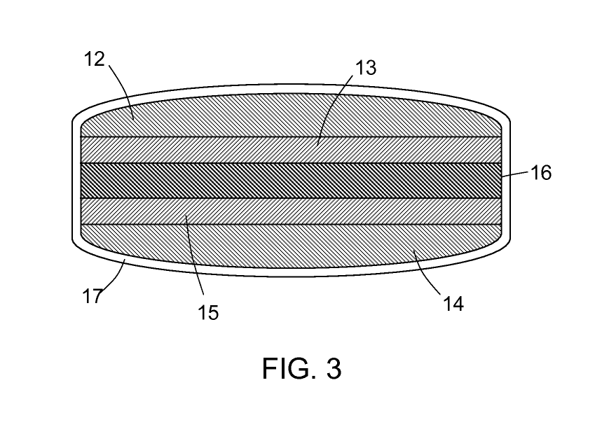

In another embodiment, the capsule of the invention may be constructed as shown in FIG. 3. The different inner bodies may be assembled in layers 12, 13, 14, 15, and 16 and the assembly may be covered with a coating 17. The coat dissolution rate is driven by the constituents and thickness of any capsule coating, the matrix, and any other optional layers of the capsule that affect the ingredient-release profile.

In yet another embodiment, the capsule incorporates pharmaceutical compounds, vitamins, herbal remedies, or other compounds used for a medical treatment. The thickness of the coating 17 and its constituents contribute to the ability to delay release of the compounds sequestered in the inner bodies. The capsule size, coating and matrix thickness, and other properties can be adjusted to achieve delayed release profiles appropriate for oral deliver of compounds to children, adults, or veterinary patients, based on their digestive systems and digestive profiles. Such compounds delivered via oral administration in a capsule placed in a liquid beverage can include cold medicine such as Robitussin™, digestive aids, such as Pepto-Bismol™, or any other compound that is appropriate for oral ingestion and remains active when administered via the digestive system.

In yet another embodiment, the invention is specially formulated for veterinary patients. For example, when the patient is a cat, the capsule may include taurine or catnip to promote ingestion of the formulation.

One advantage of this invention is that it resolves the problem of incompatible formulas in which formula components would not be stable or form homogeneous solutions if mixed together at manufacturing time, and therefore give the manufacturer a wide range of new options with regards to the various components that may be used. In other words, a capsule can be manufactured at one time and consumed at another time. Its components such as the shell, the matrix, or any one of the inner bodies could comprise chemicals or other compounds that lose some or all of their activity if they were combined or come in contact with each other during the manufacturing process. The solution provided by this invention involves the mixing of these constituents at the time of dissolution in a beverage, thereby avoiding or reducing concerns regarding loss of activity.

While the above description contains specificity, the reader should not construe these embodiments, drawings, and examples, as limitations on the scope of the invention. They are merely exemplifications of various embodiments thereof. Those skilled in the art will envision many other possible variations that fall within the scope of the present invention that would be within the knowledge of one of ordinary skill in the art reading this description. Accordingly, the scope of the invention is provided by this entire specification, known teachings, and the appended claims and legal equivalents, and not merely by the examples and embodiments that have been described here.

What is claimed is:

1. A capsule for the time release of ingredients in an aqueous beverage, said capsule comprising:
a) a shell composed of a material configured to be soluble in said aqueous beverage when a first predetermined condition is met;
b) a liquid, solid, paste, gel or powder matrix material exposed to said aqueous solution when said shell is fully or in part dissolved in said aqueous solution, said matrix configured to be soluble in said aqueous solution when a second predetermined condition is met;
c) inner bodies embedded or floating in said matrix, said inner bodies exposed to said aqueous solution when said matrix is fully or in part dissolved in said aqueous solution, each said inner body configured to be soluble in said aqueous solution when a third predetermined condition is met, said third predetermined condition being possibly different for each said inner body.

2. The capsule of claim 1 wherein any of said predetermined conditions is a temperature of said aqueous beverage.

3. The capsule of claim 1 wherein any of said predetermined conditions is a time duration of exposure to said aqueous beverage.

4. The capsule of claim 1 wherein any of said predetermined conditions is an amount of shaking of said aqueous beverage.

5. The capsule of claim 1 wherein said shell comprises a flavor or a color, said flavor or color being released into said aqueous beverage when said shell dissolves into said aqueous beverage.

6. The capsule of claim 1 wherein said matrix comprises a flavor or a color, said flavor or color being released into said aqueous beverage when said matrix dissolves into said aqueous beverage.

7. The capsule of claim 1 wherein any one of said inner bodies comprises a flavor or a color, said flavor or color being released into said aqueous beverage when said any one of inner bodies dissolves into said aqueous beverage.

8. The capsule of claim 1 wherein any one of said inner bodies is coated thereby allowing a control of when said any one of said inner bodies dissolves into said aqueous beverage.

9. The capsule of claim 1 wherein any of said predetermined condition is selected to allow a release of a color or a flavor according to a predetermined time profile.

10. The capsule of claim 1 wherein said shell, said matrix or any one of said inner bodies is chewable.

11. The capsule of claim 1 wherein said shell, said matrix or any one of said inner bodies is edible.

12. The capsule of claim 1 being manufactured at one time and consumed at another time, wherein said shell, said matrix or any one of said inner bodies comprise chemicals which lose some or all of their functions when mixed together at said manufacturing time and operate best when mixed together at said use time.

13. The capsule of claim 1 wherein said shell, said matrix or any of said inner body comprise chemicals selected from the group consisting of mint, vanilla, anis, coffee, chocolate, salt, pepper, lemon oil, orange oil, walnut oil and nut oils.

14. The capsule of claim 1 wherein said shell, said matrix or any of said inner body comprise pharmaceuticals.

15. The capsule of claim 1 wherein said matrix and said inner bodies are stacked in layers.

16. A timed-release capsule, which comprises:
a) a shell composed of a material soluble in liquid, which begins to dissolve in response to a first predetermined condition;
b) a liquid, solid, paste, gel, or powder matrix that is exposed to the liquid when the shell is partially or fully dissolved in the liquid, wherein the matrix becomes soluble in the liquid when a second predetermined condition is met; and
c) inner bodies that are present in the matrix, wherein the inner bodies are exposed to the liquid when the matrix is partially or fully dissolved in the liquid, and become soluble in the liquid when a third predetermined condition is met;
wherein said capsule provides for exposure and dissolution of the inner bodies in the stomach or intestines.
